(19) United States
(12) Patent Application Publication
(10) Pub. No.: US 2012/0207793 A1
(43) Pub. Date: Aug. 16, 2012

(54) Methods and Compositions for Controlled and Sustained Production and Delivery of Peroxides and/or Oxygen for Biological and Industrial Applications

(75) Inventors: Kevin Ward, Richmond, VA (US); Gary Huvard, Richmond, VA (US); Everette Carpenter, Richmond, VA (US); Gurbhagat Sandhu, Richmond, VA (US); Robert Barbee, Richmond, VA (US); Bruce Spiess, Richmond, VA (US)

(73) Assignee: Virginia Commonwealth University

(21) Appl. No.: 13/372,364

(22) Filed: Feb. 13, 2012

Related U.S. Application Data
(63) Continuation of application No. 12/226,945, filed on Mar. 9, 2009.
(60) Provisional application No. 60/800,041, filed on May 15, 2006.

(51) Int. Cl. A61K 33/40; A61K 9/14; A61P 17/02; A61K 9/00

(52) U.S. Cl. 424/400; 424/616

(57) ABSTRACT

Methods and compositions for the controlled and sustained release of peroxides or oxygen to aqueous environments (e.g. a patient's body or circulatory system, or for other applications) or non-aqueous environments, include a material coating or encapsulating hydrogen peroxide, inorganic peroxides or peroxide adducts. In the case of peroxide adducts, and particularly in one type of embodiment, the peroxide adducts should be able to permeate the material, but water, hydrogen peroxide and inorganic peroxides should be able to permeate the material. The methods and compositions that allow the release of oxygen, H₂O₂ or inorganic peroxides from peroxide adducts with movement of these moieties across a selectively permeable barrier into, preferably, an aqueous environment. In the case of hydrogen peroxide, it can be acted upon by catalase or other enzymes, or be simply degraded, or are otherwise acted upon by enzymes or catalysts embedded in the selectively permeable barrier to produce, for example, O₂. Alternatively, hydrogen peroxide or inorganic peroxides can be delivered selectively to a site of action of cleaning, disinfecting or other applications.

### BACKGROUND OF THE INVENTION

1. Field of the Invention

The invention generally relates to methods and compositions for the controlled and sustained release of peroxides (e.g., hydrogen peroxide, calcium peroxide, zinc peroxide, sodium peroxide, magnesium peroxide, etc.) or oxygen for use in biological, industrial, and other applications. The invention includes methods and compositions for the generation of oxygen from various peroxides in, for example, aqueous and non-aqueous environments including without limitation biological tissues in humans and animals; soil, lake and other environments; in tanks and reservoirs for industrial or medical applications, etc.

2. Background of the Invention

leading cause of preventable death due to traumatic injury on the battlefield is hemorrhage.^{1,2} Hemorrhage is the second leading cause of death in civilian trauma.³ Hemorrhagic shock leads to either immediate or delayed death by reducing oxygen delivery to vital organs to levels below those needed to sustain oxidative metabolism. When this occurs over a long enough period of time, the result is the production of massive oxygen debt or tissue ischemia.⁴ Obviously, the treatment of such injuries must utilize approaches which combine hemorrhage control (when possible) with restoration of adequate oxygen delivery to avoid accumulation of oxygen debt levels that are associated with immediate or delayed death.^{4, 5} Even when bleeding is controlled, restoration of oxygen delivery above critical threshold levels to maintain survival is challenging.

There is a need for improved mechanisms for providing oxygen to tissues and organs of humans and animals over an extended period of time. Sustained delivery of oxygen can also be a benefit to many non-medical applications. Similarly, there is a need for improved mechanisms for providing peroxides, including without limitation hydrogen peroxide and inorganic peroxides, over an extended period of time for both biological and industrial applications

### SUMMARY OF THE INVENTION

In an exemplary embodiment, a peroxide or oxygen producing composition is provided which includes a nanoparticulate peroxide slurried with a hydrophobic fluid. The hydrophobic liquid, which can be for example perfluorinated compounds such as to perfluorodeclin as well as a wide variety of other compounds protect the nanoparticulate peroxide from water until desired. The nanoparticulate peroxide is preferably present in crystalline form, but can also be non-crystalline, and is preferably on the order of nanometers in diameter, however, given application, the particulate can have median diameters that are sub-micron (10⁻² to 10⁻⁶ being preferred), millimeter, or even larger sizes. Upon exposure to water or other aqueous fluid which may diffuse or otherwise pass through the hydrophobic liquid to contact the nanoparticulate peroxide, hydrogen peroxide or oxygen is produced which can then be delivered to a desired environment (a wound, a polluted soil, a tank requiring sterilization, etc.). In the case of delivering hydrogen peroxide, the environment itself may include enzymes (catalase and others) which cause generation of oxygen from the hydrogen peroxide. The nanoparticulate peroxide might be freeze dried hydrogen peroxide, an inorganic peroxide (calcium peroxide, sodium peroxide, magnesium peroxide, etc.), or a peroxide adduct (compounds which include hydrogen peroxide molecules, e.g., sodium carbonate perhydrate (Na₂CO₃.1.5H₂O₂), urea hydrogen peroxide ((NH₂)₂CO.H₂O₂)(UHP), histidine hydrogen peroxide, adenine hydrogen peroxide, and alkaline peroxyhydrates (for example, sodium orthophosphorate).

In another exemplary embodiment, the peroxide or oxygen producing composition may be encapsulated in a membrane or coating which retains the composition and protects it from exposure to water or aqueous fluid until used. The membrane or coating preferably will selectively allow water (e.g., from the environment in which the composition is to be to used) to pass through (from the environment into encapsulated or coated composition), and will allow hydrogen peroxide or oxygen (which are similarly sized to water and have other similar characteristics) that is generated upon contact of the peroxide or oxygen producing composition with water to pass through (e.g., the oxygen or hydrogen peroxide (or inorganic peroxides (e.g. sodium, lithium, calcium, zinc, or magnesium peroxides)) will be directed out through the membrane or coating into the environment). However, the membrane or coating will retain the peroxide or oxygen producing composition. The membrane or coating might include catalysts such as iron and copper species, or enzymes such as catalase embedded therein or otherwise associated therewith such that if hydrogen peroxide is generated by contact of the peroxide or oxygen producing composition with water, the hydrogen peroxide will be converted or otherwise decomposed to oxygen upon traversal of the membrane or coating. In an alternative exemplary embodiment, the peroxide or oxygen producing composition will be interlaced into gauze (e.g., a bandage application) or other suitable carrier, where the carrier is preferably hydrophobic so as to allow the peroxide or oxygen producing composition which itself preferably includes a hydrophobic component (e.g., a hydrophobic liquid) co-mingle and associate with the carrier. The rate of delivery of the peroxide or oxygen may be controlled, without limitation, by the choice of hydrophobic liquid, the ratio of hydrophobic liquid to nanoparticulate peroxide (when the peroxide or oxygen producing composition is a slurry of the same), the characteristics of the membrane or coating which encases the peroxide or oxygen producing composition, or the characteristics of the carrier.

Whole body oxygen delivery can be described by the following equation:

DO₂=CO×CaO₂

where DO2 stands for oxygen delivery or the volume of oxygen delivered to the systemic vascular bed per minute. It is the product of cardiac output (CO) in liters/minute, and arterial oxygen content (CaO₂) cc/dl. CaO₂ can be further defined by the equation:

CaO₂=Hb×1.36×SaO₂+(PaO₂×0.003).

In this equation, Hb is hemoglobin in gm/dl, SaO₂ is the percent saturation of hemoglobin by oxygen, and PaO₂ is the partial pressure of oxygen in arterial plasma in mmHg. The factor 1.36 is the estimate of the mean volume of oxygen (ml) that can be bound by 1 gm of normal hemoglobin when it is fully saturated (SaO₂=1.0). The factor 0.003 is the solubility coefficient of oxygen in human plasma. Thus for an average human with a hemoglobin level of 15 gm/dl and with a PaO₂ of 100 mmHg (and thus an SaO₂ of approximately 1.0), an arterial oxygen content of 20.3 ml/dl of oxygen:

CaO₂=15 gm/dl×1.36×1.0+(100×0.003)=20.3 cc/dl.

As the equation demonstrates, the amount of oxygen dissolved in plasma does not normally make a significant contribution to CaO₂. This is due to the low solubility of oxygen in plasma. DO₂ for an individual with a cardiac output of 5 /min and CaO₂ of 20 cc/dl would be 1000 cc/min.

Oxygen consumption (VO₂) is the amount of oxygen that is normally consumed by tissues and averages 250 cc/min for an adult. Since oxygen transport averages 1000 cc/min, about 750 cc/min returns to the right heart in venous blood each minute. This 750 cc/min of oxygen is still carried in 5 liters or 50 dl of blood each minute. Each 1 dl therefore carries 15 cc/dl (750 cc/min divided by 50 dl/min). Thus the average VO₂ is 5 volume %.

The above discussion illustrates the challenges in restoring and maintaining tissue oxygenation in the setting of hemorrhagic shock, even when hemorrhage is controlled. Because hemoglobin is the major carrier of oxygen, simple restoration of circulating volume will, in and of itself, be insufficient to overcome reductions in CaO₂ since current intravenous fluids cannot carry oxygen any better than plasma. This problem is compounded if victims have respiratory insufficiency and cannot be provided supplemental oxygen. While these latter issues are more readily resolved in the civilian trauma setting, their recognition and correction in the combat setting can be impossible since the provision of supplemental oxygen and the routine performance of endotracheal intubation or other forms of respiratory support is severely limited. Thus hypoxemia can be a major contributing factor to critical reductions in DO₂.

Acute soft tissue wounds and burns require sufficient oxygen delivery to maintain cellular viability and to prevent superinfection. Oxygen delivery to wounds and burns is many times insufficient due to circulatory compromise from causes ranging from anemia, tissue edema, and vascular destruction. The timing and type of fluid resuscitation after incurring burns can influence the transition of partial thickness burns to full thickness burns.⁷ Therefore, metabolic support prior to definitive treatment can be tissue sparing.

Various strategies have been proposed and many studied as a means to improve short-term survival in the setting of traumatic shock. These have focused on providing low volume plasma expanders such has hypertonic saline and hetastarch as a means of increasing cardiac output and keeping tissue vascular beds open.^{8, 9} While this is helpful and tissue oxygen delivery will be improved to some extent, it cannot routinely compensate for major reductions in CaO₂ for the reasons above. Additional strategies have involved the creation of hemoglobin and nonhemoglobin based oxygen carriers (HBOC and NHBOC). While promising both HBOC's and NHBOC's have their limitations. For HBOC's, the major concern is the amount needed to raise hemoglobin to significant levels as well as storage and product source (bovine, etc).¹⁰ Even if provided in sufficient levels, hypoxemia due to various causes (inability to manage the airway, inability to provide supplemental oxygen, etc) would limit its potential ability to restore tissue oxygen delivery.

The major NHBOC strategies involve the use of perfluorocarbons (PFC's).¹⁰⁻¹² PFC's are composed entirely of carbon and fluorine. They are biologically and pharmacologically inert. PFC's have the unique ability to dissolve and carry significant quantities of gases. In terms of oxygen, PFC's have the ability to carry between 5-18 volume % (250 cc or greater of oxygen). This amount of oxygen is capable of meeting the metabolic demands of an adult human. Animal studies have demonstrated the ability of animals to survive complete exchanges of blood for PFC. However, in order for PFC's to carry large quantities of oxygen, the inspired concentration of oxygen must be very high. This would limit them in situations such as the battlefield where supplemental oxygen would not be readily available or in which the lungs were damaged and alveolar diffusion of oxygen is limited.

A recent iteration on the use of PFCs for oxygen delivery has been noted with the dodecafluoropentane (DDFP) emulsions.^{13, 14} This PFC undergoes a phase transition from liquid to gas at 37° C. (body temperature). The transition in blood leads to the development of microbubbles. These microbubbles are capable of carrying enormous amounts of gas including oxygen. Preliminary studies have demonstrated that it might be possible for as little 2-5 cc of DDFP to carry enough oxygen to meet the metabolic demands of the body. Issues with this approach include the unknown life-span of the bubbles as well as preventing phase transition prior to administration. Proper airway management and threshold levels of alveolar diffusion of oxygen would still be required, potentially limiting their value in the ultraearly stages of casualty treatment.

Neither current HBOC nor NHBOC products may impact on initial burn or wound treatments to prevent ischemia or transition to states beyond repair in the initial stages of casualty care.

In summary, there is still a technological gap in restoring and/or preventing tissue ischemia in the setting of traumatic shock and traumatic wounds, especially in austere environments such as exist on the battlefield. A need continues to exist in developing novel therapeutic approaches that enhance tissue oxygen delivery especially in the first critical hours after injury.

A standard, off-the-drugstore-shelf, 3% solution of H₂O₂ contains 30 mg H₂O₂/ml of solution, which is equivalent to 0.88 moles/l solution since the molecular weight of H₂O₂ is 34.0. Given that one mole of O₂ and two moles of H₂O are produced when two moles of H₂O₂ are exposed to the enzyme catalase, 2H₂O₂→2H₂O₂, 0.44 moles of O₂, or equivalently, 11.2 liters of O₂, are generated from one liter of this off-the-shelf H₂O₂ solution. The estimate of the volume of O₂ is made with the Ideal Gas Law (V=nRT/P, where n is the number of moles, R is the gas constant, T is the temperature in K, and P is the pressure in atm.) The normal body temperature is assumed to be 37° C. at one atm for this calculation. The consumption rate of this H₂O₂ solution is only 22 ml/min to meet the oxygen requirement of a resting 70 kg male, which is approximately 250 ml/min (˜3.6 ml/kg/min).

This large production (sometimes hyperbaric amounts) of oxygen from small amounts of H₂O₂ is attractive for medicinal uses. In fact, this relationship has been studied for medical purposes dating for the early and mid-1900s in animals and humans.¹⁵⁻²¹ Remarkable reports exist of H₂O₂ being used to resuscitate animals in cardiac standstill due to hypoxemia and coronary artery occlusion.^{<}It has also been used in an attempt to oxygenate patients with severe hypoxemia secondary to influenza.²² While reports were encouraging, these studies do not contain detailed experimental design information and proper controls. It appears that the ability to raise tissue oxygenation levels is less impressive when H₂O₂ is delivered intravenously as opposed to intra-arterially. This probably has to do with the rapid conversion of H₂O₂ in the blood to oxygen, which is then off-gased via normal ventilation.

Most reports, however, ignore the dangers of intravascular administration. It is likely that many unreported deaths have occurred due to its use. When H₂O₂ is given directly in quantities needed to raise tissue oxygenation, hyperbaric amounts of oxygen are produced. Given the low solubility of oxygen in plasma (0.3 cc/dl blood), the rapid increase in plasma oxygen levels will exceed the ability of the plasma to dissolve it particularly if hemoglobin is already fully saturated with oxygen. The result will be that the oxygen produced by H₂O₂ will come out of solution forming bubbles. These bubbles will coalesce and be capable of blocking both large vessels as well as the microvasculature. In essence a form of decompression illness will occur. Thus instead of providing oxygen to tissues, ischemia is produced in tissue beds by blockage of blood flow.

Even now, sporadic reports of death after oral ingestion of H₂O₂ exist.²³ These deaths are caused by the development of large oxygen gas emboli which occur as the result of large oxygen production in the lumen of the intestines. This rapid gas production breaches various vascular plexi in the intestines which leads to introduction of gas into the systemic circulation. Thus the use of H₂O₂ in its native form is too dangerous to contemplate its use in humans due to the uncontrolled release of oxygen. It use in hemorrhagic shock would represent an even more dangerous proposition given the concurrent loss of hemoglobin which acts as the native carrier of oxygen.

In an attempt to control the release of oxygen from the reaction of H₂O₂ with catalase in the blood, the use of urea-hydrogen peroxide (UHP) has been suggested.²⁴ UHP is a 1:1 adduct of urea and H₂O₂ and is very stable, decomposing at a temperature of 75-85° C. It is 32% H₂O₂ by weight with a density of 1.4 g/cc. One gram of UHP (32% H₂O₂ by weight and equal to 1 cc), will produce 114 cc oxygen. In this setting, the urea adduct is cleaved from the H₂O₂. The H₂O₂ is then free to react with catalase to produce oxygen and water.

UHP has been used to treat hypoxemic rabbits with some success.²⁴ However, only enough UHP was used to raise arterial PO₂ levels by 10 mmHg. Although this is a small amount, the use of UHP did allow for a rise in arterial PO₂ when given intravenously likely due to the delayed conversion of H₂O₂ into oxygen by the required cleavage of urea from the H₂O₂. However, other attempts to use UHP in amounts that would supply the oxygen consumption needs of a rabbit failed. When used in amounts necessary to do this, animals died of gas emboli. Even when used in conjunction with PFCs the amount of oxygen produced over short time periods overwhelmed the ability of the PFC to dissolve the oxygen. Use of either straight H₂O₂ or UHP in wounds would also result in conversion to O₂ at rates so rapid as to require amounts of agents too large and application times too often to be practical.

Thus, even though UHP provides a stable source of releasable oxygen in solid form with some delay in the conversion process, it is not sufficient by itself to act as the sole entity for controlled release and delivery of oxygen in amounts required to meet the metabolic needs of the body as a whole or the needs to wounds.

Many other medical and non-medical uses for the safe, controlled and sustained delivery of oxygen also exist. For example, various disinfecting, cleaning, soil cleanup, and whitening agents could benefit from advances in such technology.

Gibbons et al. (U.S. Pat. No. 7,160,553) provides matrices/dressings for oxygen delivery to tissues. However, the matrices/dressing are useful only for localized delivery of oxygen directly to tissues, e.g. directly to a wound. Gibbons also does not disclose a prolonged controlled delivery method.

Montgomery (U.S. Pat. No. 7,189,385) describes tooth whitening compositions that comprise a peroxide source. However, the compositions described by Montgomery are for external application only, and are not suitable for sustained, controlled internal oxygen delivery.

The prior art has thus-far failed to supply a viable solution to the long-standing problem to how to safely deliver large amounts of oxygen to aqueous and nonaqueous environments in a safe, controlled and sustained manner. The present invention provides compositions and methods to safely release oxygen in an aqueous or nonaqueous environment, such as in a patient's body or in non-biological applications, in a sustained, controlled manner.

The prior art also does not provide a mechanism for delivering peroxides to aqueous and non-aqueous environments over a sustained period.

According to an embodiment of the invention, a peroxide or oxygen producing composition which is encapsulated or coated with a selectively permeable material may be used to sustainably provide peroxides (e.g., hydrogen peroxide or inorganic peroxides) over an extended period of time. The peroxide or oxygen producing composition preferably includes a nanoparticulate peroxide slurried with a hydrophobic fluid. In some applications, the membrane or coating may not be present, as the hydrophobic fluid serves to keep water or other aqueous fluid from interacting with the peroxide until desired (i.e., diffusion of water into contact therewith). Also, in some applications, the peroxide or oxygen producing composition might simply include a peroxide adduct which is encased by the encapsulating material or coating. The peroxide or oxygen producing composition can be simply be placed where sustained delivery of peroxides (hydrogen peroxide or inorganic peroxides) or oxygen is desired (e.g., in a wound (e.g., use on a bandage or in a lotion or emulsion or other formulation applied thereto), in soil, in a tank (e.g., for sterilization, etc.). Upon exposure to water or other aqueous fluid which may diffuse or otherwise pass through the hydrophobic liquid (when employed) and or the encapsulating material or coating to contact the peroxide or oxygen producing moiety, hydrogen peroxide, inorganic peroxides or oxygen is produced which can then be delivered to the desired environment. The rate of delivery can be varied in a number of ways including choice of the hydrophobic liquid, varying the ratio of the hydrophobic liquid to nanoparticulate peroxide, choice of the material for encapsulation or coating, or choice of substrate which the composition is associated with. In medical treatments, the patient might be given a bolus dose of perfluorocarbon or like compounds to reduce the chance of embolism or of catalase or other enymes to supplement the generation of oxygen from hydrogen peroxide, or of oxygen scavengers to prevent oxidative damage, etc. In some applications where the peroxide or oxygen producing composition produces hydrogen peroxide, the encapsulating or coating material may have iron catalysts, catalase or other enzyme catalysts embedded therein or associated therewith to convert hydrogen peroxide to oxygen as the hydrogen peroxide traverses the membrane or coating.

### BRIEF DESCRIPTION OF THE DRAWINGS

FIG. 1A-D. Schematic representations of an embodiment of the invention. A, H₂O₂ adduct (it being understood to include any peroxide adduct which releases hydrogen peroxide or inorganic peroxides) is encapsulated or coated by a selectively permeable membrane/barrier; B, H₂O₂ adduct is embedded in a selectively permeable membrane/barrier; C, adduct-barrier mix is layered; D, adduct-barrier mixture surrounds aqueous environment.

FIG. 2A-B. Schematic representations of an embodiment of the invention in which a hydrophobic fluid surrounds the H2O2 or H₂O₂ adduct. A, H2O2 or an, H₂O₂ adduct is suspended in hydrophobic fluid, and this mixture is contained within the selectively permeable barrier, and the aqueous environment surrounds the adduct complex; B, H2O2 or H₂O₂ adduct is suspended in hydrophobic fluid, and both are separated from the aqueous environment by a selectively permeable barrier, all components being present in a layered arrangement.

FIG. 3. Oxygen delivery rates from UHP-containing microcapsules predicted from the transport model. The calculations are performed at 37° C. and 1 atm assuming 5 micron diameter microspheres with a PLGA shell thickness of 0.2 microns. The paste consists of a perfluorocarbon carrier having a maximum of 1000 ppmw of soluble water. The paste contains 60 vol % of UHP particles with sphere equivalent diameters of (A) 100 nm, (B) 200 nm, (C) 300 nm, and (D) 500 nm. Curve (E) is the predicted oxygen delivery rate from a carrier solvent paste having a UHF particle size distribution of 5 wt % (A), 5 wt % (C), and 90 wt % (D). Curve (B) illustrates the delivery of >200 cc O₂/min for more than 30 minutes and curve (E) illustrates thc delivery of ˜100 cc O₂/min for almost 1.5 hours. A total of 176 g UHF is consumed in each case.

FIGS. 4A and B. The permeation cell. A, side view; B, top view where the viewer is looking down into the permeation cell through the clear water phase in the top half of the cell. The white UHP crystals in the bottom half of the cell are visible. Also visible are the white, magnetically driven stir bars in both halves of the cell used to maintain uniform concentrations in each phase.

FIG. 5 is a plot of the experimental release of hydrogen peroxide that has diffused across the membrane in the permeation cell, compared to the release predicted by a transport model.

FIG. 6. Schematic of a hydrogen peroxide delivery microcapsule. The 2-to-5 μm diameter microcapsule contains 100-500 nm urea hydrogen peroxide particles suspended in a biocompatible perfluorocarbon. The microcapsule shell is a 0.2 μm thick poly(lactide-co-glycolide) polymer membrane.

FIG. 7. Sequence of events leading to release of hydrogen peroxide and then oxygen into the blood stream.

FIG. 8. Schematic drawing showing the process steps using an emulsion technique using high-energy homogenization to shear peroxide adduct grains into submicron particulates.

### DETAILED DESCRIPTION OF THE PREFERRED EMBODIMENTS OF THE INVENTION

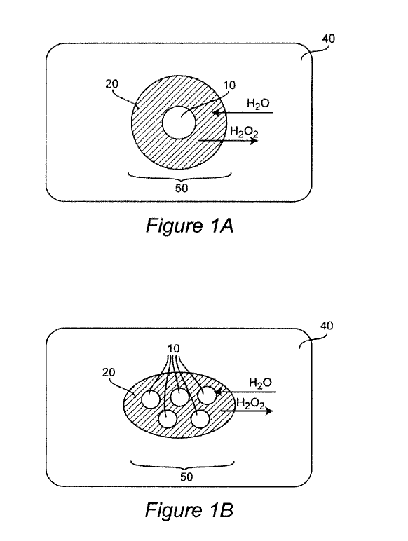

FIGS. 1a and 1b show embodiments of the invention where a peroxide or oxygen producing composition 10, which can optionally include a selectively permeable membrane or coating material 20 so as to form a complex 50 is positioned in an environment of interest 40. The environment 40, which may be aqueous or non-aqueous. Water or other aqueous fluid, which may come from the environment itself (exudate from a wound, water in the soil, etc.) or be supplied from an external source (not shown) is permitted to selectively pass through the permeable membrane or coating material 20 of the complex 50 and to come into contact with the peroxide or oxygen producing composition 10. In some embodiments, interaction of the peroxide or oxygen producing composition 10 with water, hydrogen peroxide is produced and hydrogen peroxide is permitted to pass through the material 20 or otherwise be delivered to the environment 40. In the environment 40, enzymes (e.g., catalase) or other catalysts (e.g., iron) which are naturally present or which are supplied by an external source (e.g., supplying a patient (human or animal) with additional catalase to that which is already present naturally) could be used to convert the hydrogen peroxide to oxygen. Furthermore, the membrane or coating material 20 might be constructed to include catalysts such as catalase or iron embedded therein or otherwise associated with the surface such that hydrogen peroxide which is generated by the peroxide or oxygen producing composition may be converted to oxygen as it traverses or otherwise passes through the material 20. In other embodiments of the invention, the hydrogen peroxide itself may be desired (e.g., for disinfecting a wound or industrial surface or soil sample), and the environment 40 would not necessarily include catalysts for generating oxygen from hydrogen peroxide. In still other embodiments, the peroxide or oxygen producing composition 10 will produce oxygen directly (e.g., calcium or magnesium peroxide).

As shown in FIG. 1a, the complex 50 can consist of a single granule or particle of membrane or coated peroxide or oxygen producing composition 10. However, FIG. 1 shows that a number of particles of the peroxide or oxygen producing composition 10 might be included in a complex. The diameter of the peroxide or oxygen producing composition 10, as well as the complex 50, can vary widely depending on the application. For example, in intravascular or lung delivery applications, the diameter may have a size of 5-10 μm or less. However, in wound coverings, devices which are associated with organs or tissues, or in applications which are used for other environmental, biological or industrial purposes (e.g., formation of oxygen or peroxide in tanks, formation of oxygen or peroxide in soil, formation of oxygen or peroxides for teeth whitening), the diameter can be on the order of millimeters or more.

The peroxide or oxygen producing composition 10, in a preferred embodiment, includes a nanoparticulate peroxide slurried with a hydrophobic fluid. The slurry can be produced by, for example, ball milling a perfluorocarbon (PFC) such as perfluorodeclin with a peroxide adduct such as UHP. The ball milling process can be performed in the presence of a supercritical fluid such as supercritical carbon dioxide so as to enhance the formation of a fluidized powder of the PFC and the peroxide adduct. In a preferred embodiment the UHP is present in crystalline form with the PFC. Ball milling produces nanoparticles of the UHP/PFC composition 10, and assures a close association of the UHP and PFC. The PFC is present in the form of a hydrophobic liquid and will slow down or otherwise impede water from being exposed to the UHP until the composition is placed, for example, in an aqueous environment such as in a wound where water passes through or otherwise displaces the hydrophobic liquid and comes into contact with the UHP crystals, for example. Other procedures and materials can be used to make nanoparticulate peroxide slurried with a hydrophobic fluid. For example, non-PFC hydrophobic liquids could be used; other peroxide adducts, freeze dried hydrogen peroxide, or inorganic peroxides could be used; and high pressure mixing systems could be used.

By “hydrophobic liquid”, we mean a fluid that will dissolve less than 1% by weight of water if exposed to liquid water or saturated water vapor at room temperature. Examples of suitable hydrophobic fluids include but are not limited to chlorocarbons, (methylene chloride, chloroform, carbon tetrachloride, etc.), hydrofluorocarbons (dihdrodecaflouropentane(VentrelFX)), hydrochlorofluorocarbons (e.g., HCFC 141b and HCFC 123), olefinic waxes and oils, microcrystalline waxes, silicone oils, waxes and gels, perfluorocarbons (e.g. perfluorodecalin, perfluorooctyl bromide); hydrocarbons (e.g. pentane, hexane, etc.); long chain (e.g. greater than about 600) polyethylene glycols (PEGs); ethyl acetate; various oils such as cod liver oil; glyceryl triacetate; water solubility enhancers (e.g. urea, salts, perfluorocarbon ketones, etc.); blood substitutes such as perfluoro-t-butyl cyclohexane and perfluorooctyl bromide; hydrophobic solvents (see, e.g., Flick Industrial Solvents Handbook, 3^{rd} ed., Noyes Data Corporation, Park Ridge, N.J.); etc. Solubility enhancers can also be included including without limitation 1-perfluorohexyl-3-octanone, 1-perflourooctylactanone, 1-(4-perfluorobutylphenyl)-1-hexanone, 1-hexyl-4-perfluorobenzene, and perfluoroethyl phenyl ketone. In some applications, a hydrophobic material that is not a liquid (e.g. a gel or solid) might be used in place of the hydrophobic liquid. Examples of such hydrophobic materials include but are not limited to polymers such as olefinic, styryl, and vinyl polymers, polyamides, polyesters, polyurethanes, polycarbamates, poly ether ether ketones, silicon polymers, polysilanes, fluoropolymers, olefinic and polyethelyene waxes, animal fats, gels made by dissolving polymers in hydrophobic solvents (e.g., PS in toluene, PC in MeCl₂).

When the peroxide or oxygen producing composition 10 takes the form of a nanoparticulate peroxide slurried with a hydrophobic liquid or material, the choice of hydrophobic liquid can vary widely, with PFCs being only one example. The nanoparticulate peroxide is preferably present in crystalline form, but can also be non-crystalline, and is preferably on the order of nanometers in diameter, however, given application, the particulate can have median diameters that are sub-micron (10⁻¹² to 10⁻⁶ being preferred), millimeter, or even larger sizes.

The peroxide or oxygen producing composition 10 might be interlaced into gauze or other cellulose containing materials or otherwise be associated with a carrier having a hydrophobic surface or region. For example, a bandage or wound care device may have the peroxide or oxygen producing composition 10 associated with cellulose polymers or hydrophobic surfaces or regions such that when the bandage or wound care device is applied to or inserted into a wound, it can supply, for example, hydrogen peroxide, inorganic peroxides or oxygen directly to the wound.

The peroxide adducts produce hydrogen peroxide; however, calcium or sodium carbonates or peroxides will produce oxygen directly on contact with water. In a number of embodiments of the invention the peroxide or oxygen producing composition 10 is a peroxide adduct. UHF is particularly attractive since the urea produced is physiologically compatible with the body. However, in some embodiments, freeze dried hydrogen peroxide or inorganic peroxides might be used. In most medical applications, it will be desirable to select an oxygen producing or hydrogen peroxide producing compound for use as or with the peroxide or oxygen producing composition 10.

The rate of hydrogen peroxide, inorganic peroxide or oxygen generation can be controlled by the selection of the hydrophobic liquid or by the controlling the ratio of the hydrophobic liquid to peroxide adduct. However, the rate can also be controlled by using a encapsulating or coating material 20. The membrane or coating material 20 preferably will selectively allow water (e.g., from the environment in which the composition is to be used) to pass through (from the environment into encapsulated or coated composition), and will allow hydrogen peroxide or oxygen (which are similarly sized to water and have other similar characteristics) that is generated upon contact of the peroxide or oxygen producing composition with water to pass through (e.g., the oxygen or hydrogen peroxide (or inorganic peroxide) will be directed out through the membrane or coating material 20 into the environment 40). However, the membrane or coating material 20 will retain the peroxide or oxygen producing compound separate from the environment 40 a length of time desired (e.g., until the material 20 biodegrades). In some applications, the rate of delivery will produce a flux of approximately 1-5×10⁻⁶ moles peroxide/square centimeter.

By “selectively permeable membrane” or “selectively permeable barrier” we mean that the material 20 is of a nature that allows certain molecules to pass through it by passive diffusion, while excluding others, and/or that allows the passage of different molecules at different rates. The rate of passage is dependent on the pressure, concentration and temperature of the molecules that are traversing the barrier. Such barriers are also referred to as “partially permeable” or “differentially permeable”. According to the present invention, the peroxide adduct itself should not cross the barrier in most applications. Examples of materials that are suitable for use as selectively permeable membranes/barriers include but are not limited to: poly(lactic-co-glycolic acid) (PLGA) blends (e.g. pure polyglycolic acid (PGA), pure polylactic acid (PLA), and blends in the range of about 1:100 PGA to PLA or 1:100 PLA to PGA, or various blends with ratios in between e.g. about 10:90, 20:80, 30:70, 40:60 or 50:50, the composition being known to affect crystallinity and solubility and the transport rate of water and thus of H₂O₂; polyanhydrides; polysaccharides; polyamide esters; polyvinyl esters; polybutyric acid; poly(R)-3-hydroxybutyrate, poly(c-caprolactones); etc. Preferably, and particularly when the invention is used to treat patients (humans or animals), the membrane/barrier material is non-toxic and biodegradable. Exemplary biodegradable polymers for use in human and animal patients include without limitation poly(a-hydroxy esters) including poly(glycolic acid) polymers, poly(lactic acid) polymers, poly(lactic-co-glycolic acid) co-polymers, poly(E-caprolactone) polymers, poly(ortho esters), polyanhydrides, poly(3-hydroxybutyrate) copolymers, polyphosphazenes, furnarate based polymers including poly(propylene fumarate), poly(propylene fumarate co-ethylene glycol), and oligo(poly(ethylene glycol) fumarate), polydioxanones and polyoxalates, poly(amino acids), and pseudopoly(amino acids).

In some applications of the invention, the peroxide or oxygen producing composition 10 is simply a peroxide adduct, straight hydrogen peroxide (e.g., in freeze dried form), or an inorganic peroxide (as opposed to a peroxide adduct slurried together with a hydrophobic liquid), and the peroxide adduct is coated with the selectively permeable material 20.

The present invention provides compositions and methods to safely generate or release oxygen or peroxides (hydrogen peroxides or inorganic peroxides) in aqueous and nonaqueous environments in a sustained, controlled manner. In the case of oxygen release, the source of the O₂ can be H₂O₂ which is subsequently catalyzed by exposure to iron or catalase or other enzymes to produce oxygen; a peroxide adduct; an inorganic peroxide, peroxide which directly decomposes to form oxygen, etc. The oxygen or peroxide producing compounds can be peroxide adducts such as UHP, carbamide peroxide, histidine hydrogen peroxide, adenine hydrogen peroxide, sodium percarbonate, and alkaline peroxyhdrates; inorganic peroxides such as sodium, lithium, calcium, zinc or magnesium peroxides; straight or freeze dried hydrogen peroxide. The environment 40 (i.e., the “use environment” or “aqueous environment”) can vary widely and can serve as a source of water for reaction with the H₂O₂, inorganic peroxides, or a peroxide adduct and as a recipient of the H₂O₂ or inorganic peroxides that are generated by the reaction of water (or other (e.g., non-aqueous) fluid) with the peroxide or oxygen generating composition 10. As noted above, the environment 40 may contain the enzyme catalase or other enyzmes, either naturally (e.g. when the environment is a within a patient) or through the addition of catalase or other enzymes or a source of catalase or other enzymes (e.g. when the invention is practiced outside the context of the direct treatment of patients, or when it is necessary or beneficial to augment a patient's normal supply of catalase). In some embodiments, this external environment does not contain catalase, but serves as a reservoir to hold the H₂O₂ that is generated. The H₂O₂ may then be transferred to another location at which catalase, or other agents which can liberate O₂, are present and O₂ is formed. These may include such catalysts as ferric chloride, cupric chloride, etc. By “catalase” we mean the well-known catalase enzyme found in living organisms. Catalase catalyzes the decomposition of hydrogen peroxide to water and oxygen. This enzyme has one of the highest turnover rates for all enzymes; one molecule of catalase can convert millions of molecules of hydrogen peroxide to water and oxygen per second. The enzyme is a tetramer of four polypeptide chains, each over 500 amino acids long. It contains four porphyrin herne (iron) groups which allow the enzyme to react with the hydrogen peroxide. The optimum pH for catalase is approximately neutral (pH 7.0), while the optimum temperature varies by species. In the practice of the present invention, preparations of the enzyme, as are known in the art, may be utilized. Alternatively, in some embodiments, the use of a source of catalase, (e.g. a vector that encodes the enzyme, or an organism that is genetically engineered to overproduce the enzyme) may be appropriate. Furthermore, in some application agents other than catalase which are capable of liberating O₂ may be included or added to the environment 40 However, as discussed above, it should be understood that rather than using catalase or other enzymes, the membrane itself could be fabricated to include iron or copper catalysts, and that the peroxide would be converted to oxygen as it traversed the membrane. Furthermore, it should be understood that in some applications release of hydrogen peroxide or inorganic peroxides alone is the objective (not generation of oxygen). For example, the peroxides can serve as cleaning and disinfecting agents in industrial and soil applications. In these cases, enzymes are not required. Also, it will be understood that, if oxygen generation is desired, this can be acheived by decomposition of peroxides as opposed to requiring enzymes.

The arrangement and form of the peroxide or oxygen generating composition 10 can take a wide variety of forms depending on the application. For example, the peroxide or oxygen producing composition 10 and surrounding material 20 (if any) may be prepared roughly in the shape of spheres of any useful size or amorphous particles of any useful size. They may be formed into various shapes such as discs, blocks, filaments, layers, cylinders (e.g. hollow tubes or solid cylinders), or molded to fit other useful and specific shapes, e.g. the interior of a particular container, or as a paste or gel for versatile application. Further, they may be “hard” or “brittle”, or they may be flexible or pliable in nature. An example of a means to produce various forms and properties would be the use of electrospinning to produce H₂O₂ or oxygen producing embedded nanofilaments for topical applications. In addition, electrospraying can be used to coat materials on the peroxide or oxygen producing composition 10.

While FIGS. 1a and 1b, show the environment 40 as surrounding the complex 50, this need not be the case. In some embodiments of the invention, only a portion of the complex 50 is in contact with the environment 40, e.g. only one “side” or “facet” of complex 50 makes contact with environment 40, such as is shown in FIG. 1c. In FIG. 1C the complex 50 is depicted, in an exemplary manner, as a “layer” juxtaposed to environment 40, which is also depicted, in an exemplary manner, as a “layer”. For example, the configuration of FIG. 1c might be used in a bandage or wound dressing where only a portion contacts the person's body. The configuration or FIG. 1C might also be used in various industrial applications. Those of skill in the art will recognize that many other structural arrangements might also be formed (e.g. complex 50 may surround the environment 40, and a means for O₂ egress 60 from the interior cavity formed by aqueous environment 40 out through the adduct complex 50 may be included, as illustrated in FIG. 1D. In FIG. 1D, the egress 60 can take the form of a conduit or opening in the complex 50 which allows O₂ generated in the complex 50 to be delivered to a location of interest through the point of egress. In general, any form or arrangement of the components of the invention may be utilized that suit the particular application, so long as the generation of oxygen or H₂O₂ and its entry into the environment 40 (with, for example, the evolution of O₂ by the enzymatic activity of catalase or other catalysts or by decomposition in the environment) is gradual and sustainable over a desired period of time. In other words, these events occur at a measured pace (concentration and time scale) suitable for the particular application.

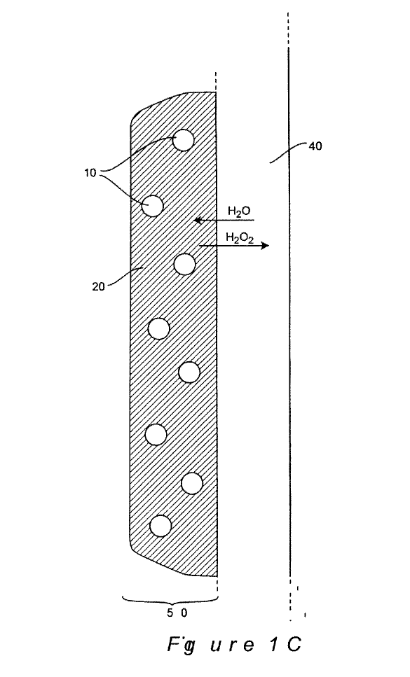

In another embodiment, a solid peroxide or oxygen generating composition can be dispersed in a hydrophobic fluid, where the mixture of the peroxide or oxygen generating composition and the hydrophobic fluid are isolated from the use environment, (e.g. an aqueous environment) by a selectively permeable barrier. This embodiment of the invention is illustrated schematically in FIGS. 2A and B. With regard to FIG. 2A, the peroxide or oxygen generating composition 10 is contained (e.g. dispersed, suspended, etc.) within a hydrophobic liquid 30 and this mixture is separated from the use environment e.g. aqueous environment 40, by selectively permeable barrier 20. FIG. 2A depicts the mixture of hydrophobic fluid 30 and the peroxide or oxygen generating composition 10 as surrounded (e.g. encapsulated or microencapsulated) by selectively permeable barrier 20, which forms a protective shell. Selectively permeable barrier 20 is in turn surrounded by aqueous environment 40. In this arrangement, complex 50 comprises the peroxide or oxygen generating composition 10, hydrophobic liquid 30 (which can be the same as or different from a hydrophobic liquid which may be slurried with nanoparticulate peroxide) and permeable barrier 20. Water diffuses from aqueous environment 40 through selectively permeable barrier 20 and thorough hydrophobic liquid 30, thereafter making contact with peroxide or oxygen generating composition 10 and causing the release of oxygen, H₂O₂ or inorganic peroxides. The released oxygen, H₂O₂ or inorganic peroxides diffuse through hydrophobic liquid 30 and selectively permeable barrier 20 into aqueous environment 40 (it being understood that the environment may be non-aqueous in some applications). In the case of an aqueous environment and where hydrogen peroxide is produced, the hydrogen peroxide is either converted to oxygen, or transported to an environment where it is converted to oxygen,

While FIG. 2A shows a permeable barrier 20 separate and apart from the hydrophobic liquid, it should be understood that in some application, the permeable barrier 20 can be dispensed with entirely. The resulting formulation having peroxide or oxygen producing composition 10 and hydrophobic liquid 30 could take the form of an emulsion when combined with water from the aqueous environment. In addition, in some applications, the hydrophobic liquid 30 could be more oil-like, or gel-like, or even a solid.

Those of skill in the art will recognize that this embodiment of the invention is not confined to the particular arrangement shown in FIG. 2A, and that many other arrangements are also possible. For example, FIG. 2B illustrates an embodiment in which the components of this O₂ generating system are laterally separated from one another and are generally present in a layer-like arrangement. Any suitable arrangement of the components may be utilized in the practice of the present invention, so long as the contact between water and the peroxide or oxygen producing composition, and the escape of generated oxygen, H₂O₂ or inorganic peroxides through the selectively permeable barrier into an environment of use, is slow enough to result in a suitably slow generation of oxygen in the environment. Furthermore, as noted above, depending on the application and the selection of hydrophobic liquid 30, the permeable barrier 20 may not be required. In addition, a hydrophobic material such as a gel or solid might be used in place of the hydrophobic liquid 30.

The oxygen generating system described herein can be used for the medical treatment of patients. It can be particularly useful for supplying oxygen to oxygen starved tissues within a patient in need thereof. The blood or plasma of the patient can be the “aqueous environment” discussed above, and can supply native catalase to convert hydrogen peroxide to oxygen. Also, the blood or plasma can be supplemented with additional catalase or other enzymes, as well as oxygen scavengers to assist in controlling the rate of oxygen generation in the patient and to prevent oxidative damage. Preferably, the peroxide or oxygen generating composition provided to the patient is in particulate form and administration may be accomplished by any of a variety of known methods, including but not limited to by injection, addition to blood or plasma being supplied to a patient, incorporation in a device or material which will contact blood or a tissue, aerosolization, ingestion, interperitoneal, intracolonic administration, administration in situ to for example explanted organs for preservation, etc. In this embodiment, the particles are preferably stored in a non-aqueous environment, e.g. “dry” such as under vacuum or with a desiccant, and are reconstituted in an administrable (e.g. liquid, emulsion, gel or solid) form prior to administration. Alternatively, the particles may be stored in a liquid material with very low or no water content (e.g. an oil or other hydrophobic liquid) and either administered directly, or further reconstituted prior to administration.

For such medical uses, such particles may be provided as an emulsion in a non-aqueous physiologically acceptable carrier such as those listed above. Of particular interest are carriers that offer the advantage of decreasing the possibility of O₂ emboli formation. Carriers such as PFCs have the ability to increase the dissolution of nonpolar gases such as O₂ (and N₂) by a factor of 20-100 fold over human plasma. As such, PFCs are known to be useful as a means of treating decompression illness, and as blood substitutes. Another suitable carrier is dodecafluoropentene. Dodecafluoropentene is capable of creating microbubbles, which may provide additional compartments within plasma to carry intravascular O₂ generated by the methods of the invention. Using the methods of the invention, an increase in the O₂ carrying capacity of the blood or plasma in the amount of at least about 1 volume percent, and preferably at least about 2 volume percent, more preferably about 3 volume percent, most preferably about 4 or even 5 volume percent or more, may be achieved. Other materials such as Crocentin which enhance diffusion through the rearrangement of water molecules may also be helpful as adjuncts.

As discussed above, although mammalian bodies contain a large amount of circulating catalase, or other agents capable of liberating O₂ medical use embodiments of the invention may also include the co-administration of additional catalase to further increase the O₂ generating capacity for the patient. In addition, other substances may be co-administered with the H₂O₂ generating material, examples of which include but are not limited to additional carriers (e.g. PFCs, blood substitutes, etc.) and antioxidants and/or free radical scavengers. Such substances may be administered in admixture with the H₂O₂ generating material (taking care to prevent excessive exposure of the H₂O₂ generating material to water during administration). Alternatively, such substances may be administered separately, sequentially (one after the other), or concomitant with administration of H₂O₂ generating material (e.g. at roughly the same time but not in the same solution or emulsion, e.g. via two intravenous lines). Delivery may be, for example: intraarterial (e.g. via catheter injection) either systemically or to isolated organ systems; intraperitoneally (e.g. via delivery to the peritoneal cavity); intrathoracic, intramediastinal, intracardiac, intrapulmonary (e.g. via injection through an intratracheal tube or via an aerosol, with or without PFCs); gastrointestinally (e.g. to stomach, intestines or colon); topically (e.g. to wounds or during surgery); intraosseously, intracystically (e.g. bladder), intracranially, intracardiac, or intranasally. The delivery of H₂O₂ generating material via non-vascular routes may be considered as a means to increase the delivery of oxygen to tissues via nonpulmonary means.

In some applications, various catalysts may be embedded into the delivery systems themselves, or molecules such as iron may be used to cause peroxides to breakdown and release oxygen.

These strategies may be useful in a wide variety of medical settings, and may be of particular use in the treatment of trauma and acute injury as a “stop-gap” measure until conventional means of providing O₂ (e.g., inhaled O₂) are available. Such scenarios include but are not limited to combat, accidents and other situations where profound shock might occur, particularly at locations remote from conventional O₂ sources. Alternatively, many other uses are also contemplated such as for treatment of asthma, pulmonary edema, acute lung injury, or airway obstruction where inhalation of O₂ is not immediately possible; or in states of extremely low blood flow such as cardiac arrest (global) or myocardial infarction, stroke, intestinal ischemia (regional) in which a large increase in oxygen content might overcome the decrease in blood flow to critical organs. Complex shock states such as sepsis (which is believed to due to a state of microvascular shunting) or states of severe tissue edema (such as burns) may also benefit by increased levels of dissolved oxygen as provided herein to overcome decreases in blood flow. Treatment of toxicologic emergencies in which oxygenation is impaired (e.g. carbon monoxide or cyanide poisoning) may also benefit from such treatment.

In terms of wound care, using the methods of the present invention, it would be possible to provide normobaric and hyperbaric oxygen externally to wounds using, for example, a special sleeve or container placed over the wound followed by addition of H₂O₂ generating material, and optionally with catalase and other catalysts and other agents or substances as described herein. This could be particularly useful in the treatment of burn victims. Wound dressings might be prepared with a hydrogen peroxide or inorganic peroxide producing material which releases peroxides slowly into a wound for use in disinfecting the wound.

Delivery of peroxides or oxygen via these methods could provide effective therapy for certain local or systemic infections by providing direct antimicrobial activity or indirectly via enhancement of the body's own immune response. The methods may also allow for development of strategies that produce whole body or regional organ preconditioning as well as allowing for the induction of significant vasodilation/hypotension to increase blood flow and thus oxygen delivery to organ systems.

Additionally, it is envisioned that certain devices could be made to take advantage of the large amounts of oxygen produced by the reaction of H₂O₂ with catalase or other catalysts. This includes creation of special containers to store harvested organs prior to transplant. In essence, a hyperbaric oxygen environment can be created in which the need for external oxygen tanks or other complex circulating equipment would not be required. H₂O₂ and other components could be added to the system to keep a hyperbaric oxygen environment present. Such a system may be able to preserve and enhance the transplantable lifetime of harvested organs. These may take the form shown in FIG. 1D, or alternatively, when no egress 60 is provided, the organ could be placed in the aqueous environment 40 that is surrounded by the complex 50. Further, application of this strategy to body cavities of organ donors (such as the intraperitoneal and intrathoracic) might assist in organ preservation until or after harvest, or, when combined with intravenous therapy, might result in the ability to create states of suspended animation. Administration in this way should also assist in systemic oxygenation.

In addition, the use of the methods of the invention need not be for dire medical emergencies. Currently, the administration of oxygen is being suggested to combat the effects of aging. Thus, small amounts of O₂ can be conveniently and safely provided to those who wish to obtain such benefits, either internally via inhalation, or by external application in washes or creams, etc.

Other methods of delivery may also be conceived, including but not limited to an external apparatus for continuous intravenous delivery in which solutions containing the maximum amount of atmospheric oxygen could be delivered based on the atmospheric pressure surrounding the patient. Thus at 1 atmosphere (760 ton), an intravenous solution of oxygen at 760 ton could be delivered by having as part of the apparatus, a means to off-gas hyperbaric amounts of oxygen prior to its entrance into the patient.

Several of the methods described above could be envisioned as useful adjunctive treatments for cancerous tumors which are known to become more sensitive to radiation therapy when exposed to higher oxygen levels. For example, a complex containing peroxide adduct or other peroxide or oxygen producing compound and/or a selectively permeable membrane can be placed in close proximity to a rumor or other tissue to oxygenate the tumor or tissue. In addition, the combination of H₂O₂ and PFC's (or other carriers) may also be useful as ultrasonic contrast agents.

The methods and compositions of the invention may also be used to produce medical grade oxygen for environments where delivery and storage of oxygen containing vessels is problematic, for example, in field hospitals or other field settings. Such a strategy would also provide other advantages, such as the simultaneous ability to purify water sources for consumption. For example, particles containing a peroxide adduct, or peroxide nanoparticles slurried together with a hydrophobic liquid or other material, and/or a selectively permeable membrane can be added to water during purification. Many other uses of the O₂ generating systems described herein are also possible.

As discussed above, the systems should also be considered as H₂O₂ generating systems, and the generation of H₂O₂ may be the primary goal. In these application, catalase and/or agents to release O₂ are avoided until desired at a later time. Examples of uses of the systems described herein, in addition to those listed above, include but are not limited to: use for delivery of hydrogen peroxide to a wound as a disinfectant; use in whitening systems, e.g. for tooth whitening or as a whitening agent in cleaning products; generation of O₂ at sites such as in aquariums or in soil (e.g. an additive to potting soil, lawns, etc.); production of a deodorizing effect, e.g. at sites on or within fabric and/or clothing inserts, in cat litter, or in products designed for application to the body; for the purpose of generating “bubbles” in a liquid for any reason; etc.).

In one exemplary application, the peroxide releasing devices (i.e., devices which use the peroxide or oxygen generating compositions described herein) can be incorporated with ferrous oxide (rust) and citric acid into recycled paper in the form of, for example, pellets. These pellets may be added to soil containing organic contaminants (e.g., gasoline, solvents, etc.). Water in the soil causes release of the peroxide to the aqueous soil environment where the peroxide is decomposed by the catalytic action of the iron and acid to create hydroxyl radicals. Hydroxyl radicals are well known oxidants for organic materials and the chemistry employed is often referred to as Fenton's chemistry. Fenton's Reagent is a combination of hydrogen peroxide with catalytic amounts of iron II or III or copper II (another catalyst which might be used in the practice of this invention), and an acid to create a pH in the range of 3-5. Hence, the present invention will generate a Fenton's reagent in situ so as to eliminate organic soil contaminants.

Production of the O₂ generating systems described herein requires that the characteristics of the various components and their interactions with each other be taken into account, as well as the particular use of the system. For systems that are used in vivo, preferably all components will be either non-toxic or used at a level at which they are non- (or only mildly) toxic, so as to avoid causing further injury to the patient. Chief among the considerations is the determination of suitable levels or rates of O₂ production, as modulated by the porosity of the selectively permeable barrier. The barrier must be sufficiently porous such that sufficient water will diffuse in and make contact with the hydrogen peroxide, inorganic peroxides, or peroxide adducts to generate a worth-while amount of O₂, but must exclude water sufficiently to prevent a burst or bursts of O₂ generation.

Various additives may be included in the material to supplement or modulate its properties. For example, solubility enhancers, oxygen scavengers, stabilizers, clarifiers, buffers, antimicrobials (e.g., parabens and benzalkonium chloride), coloring agents, etc. may be included. Furthermore, the microencapsulation technique may be modified to allow for the production of capsules which also serve to act as volume expanders by increasing the tonicity or oncocity of the injection. This may be done by decorating the capsules with certain moieties such as starches or with the use of dendrimers attached to the capsule which can carry these moieties. Inclusion of volume expanding substances within the interior of the microcapsules which are released over time might be considered. The end result is that in addition to increasing the circulating volume of oxygen, the materials also serve to expand the circulating volume of fluids within the cardiovascular systems. This leads to increases in tissue blood flow and hence oxygen delivery. Furthermore, anti-inflammatory and/or antioxidant agents might be incorporated into the delivery system either separately or as a part of the microcapsule. Dendrimers for example could be used which are highly anionic as a potential means to decrease microvascular inflammation.

The following examples serve to illustrate various non-limiting embodiments of the invention.

### EXAMPLES

### Example 1

### Development of a Transport Model

To investigate rationally the impact of the myriad of variables and focus the experimental scope of this project, we developed a transport model for the delivery process. The model allows us to simulate the oxygen delivery rate for any combination of geometric and mass loading variables and thereby design and plan the construction of a hydrogen peroxide delivery system to produce the desired amounts of oxygen. The rates of diffusion of water into the microcapsules, the rate of generation of hydrogen peroxide from the reaction of water with urea hydrogen peroxide (UHP) particles, and the diffusion of hydrogen peroxide out the microcapsules were computed using the following equations. Shrinking core kinetics were assumed for the UHP-water reaction and the UHP particles were assumed to be spherical for ease of computation. Other values for the transport coefficients, reaction rate constants, microcapsule compositions, and different particle geometries are easily incorporated. The model equations are given in dimensionless form. The model provides an efficient means to identify workable combinations of geometric and mass loading variables as targets for the experimental studies and considerably reduces the complexity of the search for a practical delivery system. Example calculations strongly support the feasibility of our approach. The model results demonstrate that readily achievable combinations of UHP size, microcapsule size, and shell thickness can be combined to produce an efficacious way to deliver hydrogen peroxide to the blood at the sustained rates needed to keep a person alive for Ito 2 hours. These results would be applicable to other H₂O₂ adducts coated with hydrophobic materials and/or permeable membranes.

The model used to simulate the hydrogen peroxide delivery process is as follows:

Rate of change of the UHP particle radius with time

$$\frac{d ( {\overline{R}}_{UHP} )}{d ( θ )} = - {N}_{Dmk} {\overline{C}}_{pgw}$$

$$θ = 0 ; {\overline{R}}_{DHP} = 1 , {\overline{C}}_{pgw} = 0$$

Rate of change of the UHP particle surface area with time

$$\frac{d ( {\overline{S}}_{p} )}{d ( θ )} = - 2 {N}_{DMK} {\overline{C}}_{pgw} {R}_{UHP}$$

$$θ = 0 ; \overline{S} - p = 1 , {\overline{C}}_{pgw} = 0$$

Mass balance on water in the perfluorocarbon carrier

$$\frac{d \overline{C} pgw}{d θ} = \frac{- 3 α}{( 1 - {V}_{px} )} \frac{δ {\overline{C}}_{pw}}{δ z} {\vert}_{zw} \\ θ = 0 ; {\overline{C}}_{pgw} = 0$$

Mass balance on water on the PLGA shell

$$\frac{δ {\overline{C}}_{pw}}{δθ} = \frac{δ 2 {\overline{C}}_{pw}}{δ z 2} + ( \frac{2 α}{α z + 1} ) \frac{δ {\overline{C}}_{pw}}{δ z}$$

$$θ = 0 ; {\overline{C}}_{pw} = 0$$

$$z = 0 ; {C}_{pw} = {k}_{wg} {\overline{C}}_{pgw} ( z = 0 is at the inner wall )$$

$$z = 1 ; {\overline{C}}_{pw} = {k}_{w} ( z = 1 is at the outer wall )$$

Mass balance on hydrogen peroxide in the perfluorocarbon carrier

$$\frac{d {\overline{C}}_{pgx}}{d θ} = φ \overline{S} p {\overline{C}}_{pgw} - \frac{3 α}{( 1 - {V}_{px} )} \frac{δ {\overline{C}}_{px}}{δ z} {\vert}_{z = 0} \\ θ = 0 ; {\overline{S}}_{p} = 1 , {\overline{C}}_{pgx} = 0 , {\overline{C}}_{pgw} = 0$$

Mass balance on hydrogen peroxide in the PLGA shell

$$\frac{δ {\overline{C}}_{px}}{δθ} = \frac{{δ}^{2} {C}_{px}}{δ {z}^{2}} + ( \frac{2 α}{α z + 1} ) \frac{δ {\overline{C}}_{px}}{δ z}$$

$$θ = 0 ; {\overline{C}}_{px} = 0$$

$$z = 0 ; {\overline{C}}_{px} = {k}_{xg} {\overline{C}}_{pgx}$$

$$z = 1 ; {\overline{C}}_{px} = 0$$

Rate of hydrogen peroxide delivery into the blood stream

$$\frac{d \overline{M}}{d θ} = γα \frac{δ {\overline{C}}_{px}}{δ z} {\vert}_{z = 1} \\ θ = 0 ; \overline{M} = 0 , {\overline{C}}_{px} = 0$$

Dimensionless parameters

$${N}_{Dmk} = \frac{( \overline{V} {k}_{rxn} {V}_{PG} {{C}_{{w}_{plasma}} ( {R}_{o} - {R}_{i} )}^{2} )}{{DR}_{UHP}^{o}}$$

$$α = \frac{{R}_{o} = {R}_{i}}{{R}_{i}}$$

$$φ = ( \frac{{k}_{rxn} {{S}_{p}^{o} ( {R}_{o} - {R}_{i} )}^{2}}{D} )$$

$$γ = \frac{3 {R}_{i} {V}_{o} {C}_{{w}_{plasma}}}{{R}_{o} {M}^{o}}$$

### Definition of Dimensionless Variables

$${\overline{R}}_{UHP} = \frac{{R}_{UHP}}{{R}_{UHP}^{o}}$$

$$S = \frac{{S}_{p}}{{S}_{p}^{o}} and {S}_{p}^{o} = 4 {π ( {R}^{o} )}^{2} {N}_{p}$$

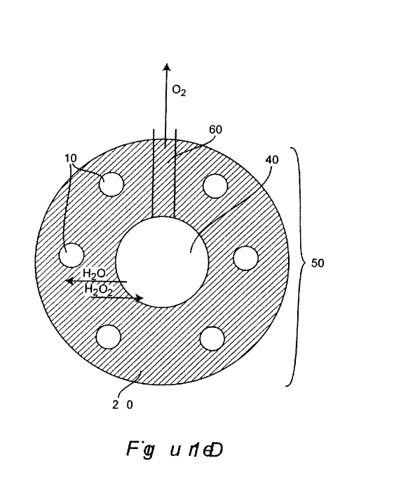

Nₚ=the total number of UHP particles in a microcapsule

$$θ = \frac{Dt}{{( {R}_{o} - {R}_{i} )}^{2}} ( dimensionless time )$$

$$z = \frac{r - {R}_{i}}{{R}_{o} - {R}_{i}} ( dimensionless distance )$$

$${\overline{C}}_{pw} = \frac{{C}_{pw}}{{C}_{{w}_{plasma}}}$$

$${\overline{C}}_{px} = \frac{{C}_{px}}{{C}_{{w}_{plasma}}}$$

$${\overline{C}}_{pgx} = \frac{{C}_{pgx}}{{C}_{{w}_{plasma}}}$$

$$\overline{M} = \frac{M}{{M}^{o}}$$

where M^{o} is the initial moles of UHP in a microcapsule Notation
V̅=molar volume of UHP (67.19 cc/mol)
MW =molecular weight of UHP (94.07 g/mol)
kᵣₛₙ=rate constant for the UHP-water reaction (400 cm⁻² see⁻¹)
V_{PG}=volume of the perfluorocarbon carrier
C_{w plasma}=concentration of water in blood plasma (˜0.055 mol/cm³)
C_{pw}=concentration of water in the PLGA shell
Cₚₓ=concentration of hydrogen peroxide in the PLGA shell
C_{pgw}=concentration of water in the perfluorocarbon carrier
C_{pgx}=concentration of hydrogen peroxide in the perfluorocarbon carrier
M=mols of hydrogen peroxide delivered from a microcapsule to the blood
R₀=outside radius of the microsphere
Rᵢ=inside radius of the microsphere
D=diffusion coefficient of water or H2O2 in the PLGA shell
R_{o UHP}=initial radius of the UHP particles inside the microcapsule
Vₚₓ=volume fraction of the UHP particles inside the microcapsule
k_{w}=partition coefficient for H2O between the PLGA shell and blood (0.011 moles water/cm3 polymer)/(moles water/cm³ in the blood)
k_{wg}=partition coefficient for H₂O between the PLGA shell and the UHP carrier
k_{tg}=partition coefficient for H₂O₂ between the PLGA shell and the UHP carrier
(k_{wg}=k_{xg} and k_{wg}=10 k_{w} was assumed for the simulations shown in FIG. 3)

Each of the elements of the proposed delivery system has been chosen after careful consideration of the oxygen delivery requirements, of the constraints imposed by human biocompatibility, of the influence of reaction kinetics, thermodynamics, and molecular transport parameters on the production and delivery of hydrogen peroxide, of the commercial availability of the various materials required, and of the feasibility of synthesizing the microcapsules. Despite what combination is chosen, the concomitant use of a perfluorocarbon carrier is indicated in order to ensure that the amount of oxygen produced by H₂O₂ delivery does not overwhelm the plasma's ability to keep the oxygen that is produced in solution (it being understood that there is a different between the internal PFC used in the oxygen or peroxide generating composition and the external PFC carrier).

PFCs are known to be able to dissolve between 5-18 vol % of oxygen. The curves in FIG. 3 illustrate the potential for achieving therapeutically useful oxygen delivery rates with different combinations of microcapsule construction. Microcapsules having a 60 vol % loading of 100 nm UHP particles in a perfluorocarbon carrier having a 1000 ppmw water saturation limit should deliver O₂ with a profile similar to Curve A. The profile in Curve B corresponds to a 60 vol % loading of 200 nm UHP particles in the fluorocarbon, curve C is for microcapsules containing 60 vol % of 300 nm UHP particles, and curve D is for microcapsules containing 60 vol % of 500 nm UHP particles. Curve E is the predicted O₂ delivery rate for a composite containing 5 wt % A, 5 wt % C, and 90 wt % D microcapsules.

Many different oxygen delivery profiles may be realized by mixing different sizes of microcapsules coated with different thicknesses of membrane materials having different rate-influencing transport properties. Consider the oxygen delivery rates shown by Curves B and E in FIG. 3. For the E simulation, microcapsules with different sizes of UHP particles were mixed to achieve a balance between a quick O₂ burst as the mixture enters the bloodstream and the longer-term delivery of O₂ supplied by the microcapsules with larger UHP particles. The E composite simulated in FIG. 3 shows an oxygen delivery rate which rises to about 100 cc/min within about 10 minutes and sustains this rate for nearly 90 minutes before slowly declining. Alternatively, the simulation of curve B used 200 nm UHP particles to deliver >200 cc O₂/min for 30 minutes starting about 10 minutes after injection.

Practically, it is quite difficult to make perfectly uniform UHP particles used in the simulation by grinding or ball milling UHP powder. Ball milling produces a distribution of sizes and the separation of ground particles by size is an imperfect art. However, it is not important that we segregate uniformly sized UHP particles in different microcapsules. If each microcapsule contains a blend of different size particles, the release behavior will be the same as for our hypothetical blend of microspheres containing segregated UHP sizes so long as the overall particle size weight fractions are reasonably the same between the two types of mixtures. The imperfect separation of particle sizes in commercial processes notwithstanding, the production of nanometer-size particle distributions is both practical and commonplace. High energy ball milling can be carried out at very low temperatures (e.g., a −10° C. glycol solution might be used to keep the material cool during grinding). For example, 20 g of UHF, 100 ml perfluorodecalin and 170 g or zirconium oxide spheres (ρ=5.68 g/ml) may be introduced into a 150 ml milling chamber under liquid full conditions where the chamber is rotated for 3-4 hours. As an alternative to ball milling, sonication, for example, high wattage sonication, might be used to produce nanoparticles

Based on a human cardiac output of 5 L/min of blood containing an arterial O₂ concentration of 8630 μmol O₂/L vs. a venous concentration of 5874 μmol O₂/L, the metabolic rate of oxygen consumption is 0.5 g O2/min. The injection of 176 g of UHP is required to generate 0.5 g O₂/min for 60 minutes. If the UHP is dispersed at 60 vol % in the perfluorocarbon carrier, 5 μm diameter microcapsules carrying a total of 176 g of UHP will occupy 237 cm³. Emergency treatment with these microcapsules would require the injection of about 500-700 cc of a 45 wt % microcapsule suspension. A 45 wt % loading corresponds to about 35 vol % in the injection mixture. According to Einstein's classical equation for the viscosity of slurries of uniform spherical particles, the viscosity of a 35 vol % suspension of 5 um diameter spheres in the water/PEG (or perfluorocarbon) mixture will be 5-6 cp. This is less than the viscosity of packed red cells which is approximately 10 cp. Thus, delivery of sufficient O₂ for a one-hour traumatic shock treatment is feasible. Additional volume strategies exists which may allow significant reduction in required injection volumes.

### Example 2

### Use of a Diffusion Cell to Measure the Generation of H₂O₂

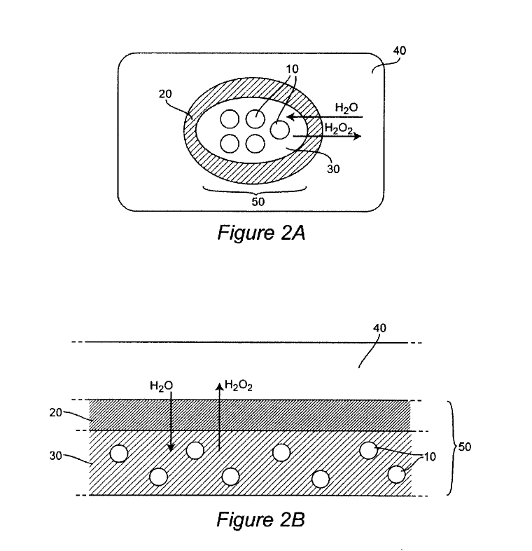

A diffusion cell was constructed in order to measure the release rate of hydrogen peroxide from UHP and its diffusion across a selectively permeable membrane. A side view of the cell is provided in FIG. 4A and a top view is provided in FIG. 4B. UHP was dispersed in a PFC liquid and maintained in the bottom half of the cell. Rather than coat the particles, a flat PLGA membrane was used to separate the UHP from distilled water located in the top half of the cell. The PLGA membrane is permeable to water and hydrogen peroxide, but is a very effective barrier to permeation of the PFC. Thus, during the experiment, water diffused across the PLGA membrane and into the PFC/UHP slurry in the bottom half of the cell. Hydrogen peroxide was generated when the water contacted the UHP. The hydrogen peroxide then diffused through the PLGA membrane into the top half of the diffusion cell.

The amount of hydrogen peroxide in the top half of the cell was monitored colorimetrically by testing samples that were periodically removed from the water-rich phase in the top half of the cell. The testing was carried out using the Ferric Thiocyanate Method (see, D. F. Boltz and J. A. Howell, eds., Colorimetric Determination of Nonmetals, 2^{nd} ed., Vol. 8, p. 304 (1978). The ferric thiocyanate method consists of ammonium thiocyanate and ferrous iron in acid solution. Hydrogen peroxide oxidizes ferrous iron to the ferric state, resulting in the formation of a red thiocyanate complex. The absorbance of the red solution obtained is measured using a colorimeter and the quantity of hydrogen peroxide required to give the absorbance can be computed.

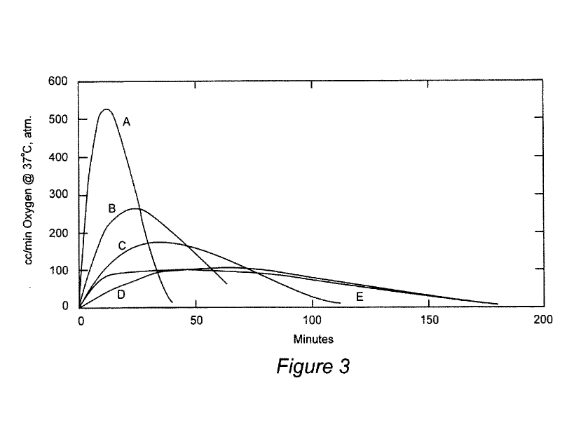

As explained, according to this test, an increase in color intensity over time correlates with an increase in peroxide concentration in the water. The results are presented in FIG. 5, where they are compared to the prediction from a transport model for microspheres that have a coating with the same thickness as the membrane used in the experiment. As can be seen, the model simulation adequately captures the actual rate of hydrogen peroxide release across the membrane, and the results validate the model and design approach. This example demonstrates the efficacy of the proposed chemistry for controlled delivery of hydrogen peroxide to, for example, the blood for oxygen production by catalase. The example also demonstrates the selectivity of the membrane and the ability to isolate the PFC and urea byproduct from the blood during hydrogen peroxide delivery. The example further demonstrates the ability to deliver hydrogen peroxide to the blood at a rate needed for tissue oxygenation.

Worth noting is that the PLGA membrane used in these preliminary experiments did not swell or rupture and the PFC and urea did not diffuse through the membrane.

### Example 3

### Microencapsulation of UHP for Intravascular Administration

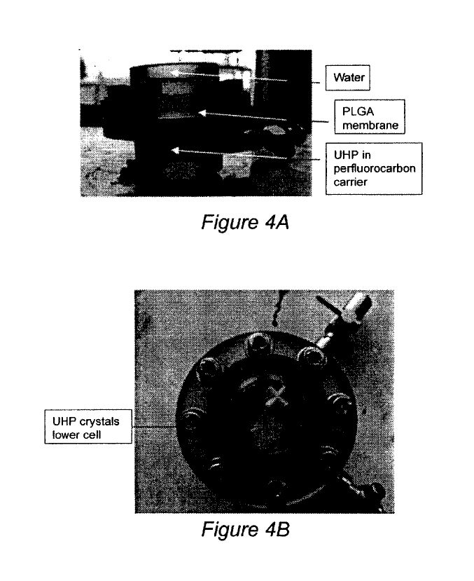

The microcapsule contains tiny particles of urea hydrogen peroxide (UHP) suspended in a biocompatible, anhydrous carrier solvent, such as perfluorodecalin. The consistency of the suspension is that of a paste. Micron-sized droplets of this paste are created in a non-solvent for the perfluorodecalin and then encapsulated with a nanometer-thick shell of biodegradable poly(lactide-coglycolide) (PLGA) copolymer. This is illustrated in FIG. 6. Encapsulating a UHP/perfluorodecalin paste mitigates the initial release “burst” of hydrogen peroxide that is anticipated to occur if UHP alone is coated. After removal of the encapsulation solvent, dry microcapsules containing the UHP/perfluorodecalin paste are recovered. The dry microcapsules are resuspended in an inert, biocompatible fluid phase (the injection carrier) for storage and transport. The susceptibility of the microcapsules to water requires storage under anhydrous conditions. High solids microcapsule pastes in anhydrous polyethylene glycol (PEG) are produced and the paste is mixed with a carrier prior to injection.

Although UHP will also react slowly with PEG, the molecular weight of PEG prevents the molecule from diffusing across the PLGA barrier at rates high enough to be problematic for long-term storage. When needed for trauma treatment, the microcapsule/injection carrier suspension is mixed with a biocompatible carrier such as PFC and injected into the blood stream.

### Example 4

### Administration of Microencapsulated UHP

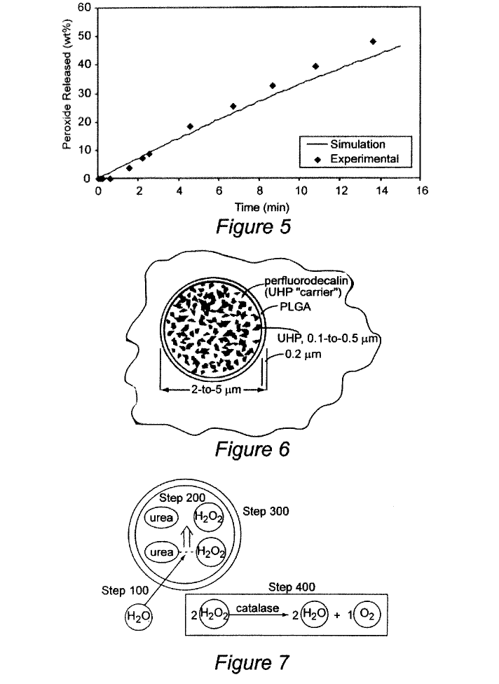

The sequence of events described next results in the generation of oxygen in the blood. The diagram in FIG. 7 illustrates the sequence of events that results in the generation of oxygen in the blood. The water that contacts the microcapsules penetrates the outer shell of the microcapsule, quickly saturates the perfluorodecalin, and attacks the UHP particles (100). Water catalytically cleaves hydrogen peroxide from the UHP adduct leaving urea as a by-product (200). One water molecule can release many molecules of hydrogen peroxide from the solid. The hydrogen peroxide also quickly saturates the perfluorodecalin and begins to diffuse through the PLGA shell, out of the microcapsule, and into the bloodstream (300). Once in the bloodstream, the hydrogen peroxide reacts virtually instantaneously with the ubiquitous catalase and releases oxygen into the blood (400).

### Example 5

### Microencapsulation of UHP by PLGA

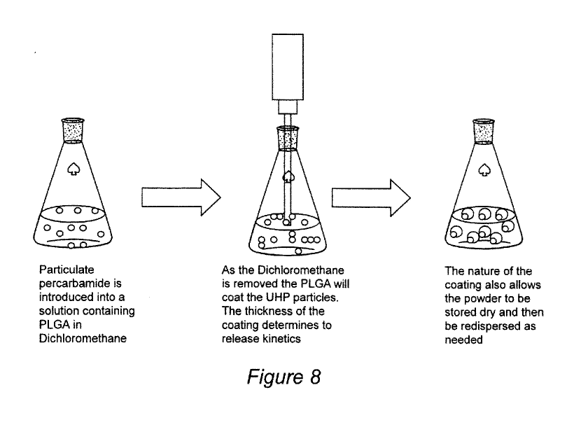

As shown by example in FIG. 8, the microcapsule contains tiny particles of urea hydrogen peroxide (UHP) coated with a biocompatible polymer such as biodegradable poly(lactide-coglycolide) (PLGA) copolymer in order to regulate the rate of oxygen production. The PLGA provides a barrier which separates the UFO solid from catalysts. As the microcapsule is introduced to a wound area or intravenously water diffuses across the barrier dissolving the UHP liberating H₂O₂ which diffuses back across the barrier. The hydrogen peroxide is quickly decomposed by available catalyst or catalyase to produce oxygen. The dry microcarrier is stable for months on end provided it is stored in a dry environment.

FIG. 8 shows the microcapsule is synthesized using an emulsion technique using high-energy homogenization to shear the UHP grains into submicron particulates from 10-900 nm in size. The 1.0 g UHP is introduced into 1.6 to 4.0 g/L solution of PLGA in dichloromethane and homogenized using an IKA T18 rotary homogenizer operating at 20,000 rpm for 25 minutes. The resulting slurry is then freeze dried to remove the dichloromethane creating the coated microcapsule which is 0.2 to 1.2 um in final size. The concentration of the PLGA in dichloromethane determines the thickness of the coating and thus controlling the release kinetics.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

1. Bellamy R. Combat trauma overview. In: Zajtchuch R, Grande C M, eds. Textbook of Military Medicine. Vol. 4. Washington, D.C.; TMM Publication, 1995:1-42.
2. Champion H R, Bellamy R F, Roberts C P, Leppaniemi A. A profile of combat injury. J Trauma 2003; 54:513-9.
3. Sauaia A, Moore F A, Moore E E, et al. Epidemiology of trauma deaths: a reassessment. J Trauma 1995; 38:185-93.
4. Rixen D, Siegel J H. Bench-to-bedside review: oxygen debt and its metabolic correlates as quantifiers of the severity of hemorrhagic and post-traumatic shock. Crit Care 2005; 9:441-53.
5. Sauaia A, Moore F A, Moore E E, Haenel J B, Read R A, Lezotte D C. Early predictors of postinjury multiple organ failure. Arch Surg 1994; 129:39-45,
6. Snyder J V. Oxygen transport: The model and reality. In: Snyder J V, Pinsky M R, eds. Oxygen transport in the critically ill. Chicago: Year Book, 1987:3-23.
7. Kim D E, Phillips T M, Jeng J C, et al. Microvascular assessment of burn depth conversion during varying resuscitation conditions. J Burn Care Rehabil 2001; 22:406-16,
8. Rhee P. Koustova E, Alam H B. Searching for the optimal resuscitation method: recommendations for the initial fluid resuscitation of combat casualties. J Trauma 2003; 54:S52-62.
9. Dubick M A, Atkins J L. Small-volume fluid resuscitation for the far-forward combat environment: current concepts. J Trauma 2003; 54:S43-5.
10. Winslow R M. Blood Substitutes. Advanced Drug Delivery Reviews 2000; 40:131.
11. Spahn D R. Blood substitutes. Artificial oxygen carriers: perfluorocarbon emulsions. Crit. Care 1999; 3:R93-7.
12. Spiess B D. Perfluorocarbon emulsions: one approach to intravenous artificial respiratory gas transport. Int Anesthesiol Clin 1995; 33:103-13.
13. Van Liew H D, Raychaudhuri S. Stabilized bubbles in the body: pressure-radius relationships and the limits to stabilization. J Appl Physiol 1997; 82:2045-53.
14. Van Liew H D, Burkard M E. High oxygen partial pressure in tissue delivered by stabilized microbubbles. Theory. Adv Exp Med Biol 1997; 411:395-401.
15. Ackerman N B, Brinkley F B. Comparison of effects on tissue oxygenation of hyperbaric oxygen and intravascular hydrogen peroxide. Surgery 1968; 63:285-290.
16. Balla G A, Finney J W, Aronoff B L, et al. Use Of Intra-Arterial Hydrogen Peroxide To Promote Wound Healing. Am J Surg 1964; 108:621-9.
17. Gaffney F A, Lin J C, Peshock R M, Bush L, Buja L M. Hydrogen peroxide contrast echocardiography. Am J Cardiol 1983; 52:607-9.
18. Jay B E, Finney J W, Balla G A, Mallams J T. The Supersaturation Of Biologic Fluids With Oxygen By The Decomposition Of Hydrogen Peroxide, Tex Rep Biol Med 1964; 22:106-9.
19. Urschel H C, Jr. Progress in cardiovascular surgery. Cardiovascular effects of hydrogen peroxide: current status. Dig Chest 1967; 51:180-92.
With Hydrogen Peroxide. Surg Forum 1964; 15:273-4,
21. Urschel H C, Jr., Morales A R, Finney J W, Balla G A, Race G J, Mallarns J T. Cardiac resuscitation with hydrogen peroxide. Arm Thorac Surg 1966; 2:665-82.
22. Oliver T, Cantar B, Murphy D. Influenzal pneumonia: the intravenous injection of hydrogen peroxide. Lancet 1920; i.
23. Moon J M, Chun B J, M M Y I. Hemorrhagic gastritis and gas emboli after ingesting 3% hydrogen peroxide. J Emerg Med 2006; 30:403-6.
24. Wu J N. Effect of urea-hydrogen peroxide on hypoxia in rabbits. Respiration 1985; 48:303-9.

1-45. (canceled)

46. A composition comprising a peroxide or oxygen producing compound together with a hydrophobic liquid or hydrophobic material.

47. The composition of claim 46 wherein said hydrophobic liquid or hydrophobic material is selected from the group consisting of chlorocarbons, hydrofluorocarbons, hydrochlorofluorocarbons, olefinic waxes and oils, microcrystalline waxes, silicone oils, waxes and gels, perfluorocarbons, hydrocarbons, polyethylene glycols (PEGs), ethyl acetate, cod liver oil, glyceryl triacetate, blood substitutes, and hydrophobic solvents.

48. The composition of claim 46 wherein said hydrophobic liquid or material is selected from the group consisting of olefinic, styryl, and vinyl polymers, polyamides, polyesters, polyurethanes, polycarbamates, poly ether ether ketones, silicon polymers, polysilanes, fluoropolymers, olefinic and polyethelyene waxes, animal fats or lipids, and gels made by dissolving polymers in hydrophobic solvents.

49. The composition of claim 46 further comprising a membrane or coating material which covers said peroxide or oxygen producing compound slurried together with said hydrophobic liquid or material, wherein said membrane or coating material permits water, hydrogen peroxide, and oxygen to pass therethrough, but prevents or delays a rate of transport of said peroxide or oxygen producing compound slurried together with said hydrophobic liquid or material through said membrane or coating material.

50. The composition of claim 49 further comprising a catalyst embedded in or associated with said membrane or coating material.

51. The composition of claim 50 wherein said catalyst includes iron or copper.

52. The composition of claim 50 wherein said catalyst includes catalase.

53. The composition of claim 46 further comprising a substrate having a hydrophobic surface or region, wherein said peroxide or oxygen producing compound slurried together with said hydrophobic liquid or material is associated with said hydrophobic surface or region.

54. The composition of claim 53 wherein said substrate is a bandage or wound care device.

55. The composition of claim 46, wherein the peroxide or oxygen producing compound is in the form of particles, which particles are slurried together with a perfluorocarbon or other hydrophobic liquid.

56. The composition of claim 55 wherein said particles have a mean diameter of less than 10p.

57. The composition of claim 46 wherein said peroxide or oxygen producing compound is freeze dried hydrogen peroxide.

58. The composition of claim 46 wherein said peroxide or oxygen producing compound is an inorganic peroxide.

59. The composition of claim 46 wherein said peroxide or oxygen producing compound is a peroxide adduct,

60. The composition of claim 59, wherein the peroxide adduct is slurried together with a perfluorocarbon.

61. The composition of claim 60 wherein the peroxide adduct is selected from sodium carbonate perhydrate, histadine hydrogen peroxide, adenine hydrogen peroxide, urea hydrogen peroxide, and alkaline peroxyhydrates.

62. The composition of claim 60 wherein said perfluorocarbon is perfluorodeclin.

63. The composition of claim 46 further comprising a membrane or coating material which covers said peroxide adduct slurried together with said perfluorocarbon, wherein said membrane or coating material permits water, hydrogen peroxide, and oxygen to pass therethrough, but prevents or delays a rate of transport of said peroxide adduct slurried together with said perfluorocarbon through said membrane or coating material.

64. A method of providing oxygen or hydrogen peroxide to a patient (human or animal) in need thereof, comprising the steps of:
a) administering to the patient an oxygen producing or hydrogen peroxide producing composition encapsulated in or coated with a material which is permeable to water, hydrogen peroxide and oxygen, and which prevents or reduces the transport of the oxygen producing or hydrogen peroxide producing composition therethrough;
b) permitting water or an aqueous fluid to pass through the material and to contact the oxygen producing or hydrogen peroxide producing composition; and
c) permitting oxygen or hydrogen peroxide generated by a reaction of the water or aqueous fluid and the oxygen producing or hydrogen peroxide producing composition to pass through the material to come into contact with the patient or a device associated with the patient.

65. A method of providing hydrogen peroxide, inorganic peroxide, or oxygen to an environment of interest, comprising the steps of:
a) positioning a composition comprising a peroxide adduct, inorganic peroxide, or freeze dried hydrogen peroxide slurried together with a hydrophobic liquid or material in proximity to or communication with an environment in which hydrogen peroxide, inorganic peroxide, or oxygen is desired; and
b) exposing the composition to water or aqueous fluid so as to generate one or more of hydrogen peroxide, inorganic peroxide, or oxygen from said composition.
